(19) United States
(12) Patent Application Publication
(10) Pub. No.: US 2019/0093046 A1
(43) Pub. Date: Mar. 28, 2019

(54) STABLE FRESHENING COMPOSITIONS AND PRODUCTS COMPRISING THE SAME

(71) Applicant: The Procter & Gamble Company, Cincinnati, OH (US)

(72) Inventors: David TURNER, Cincinnati, OH (US); Judith Ann HOLLINGSHEAD, Batavia, OH (US); George Kavin MORGAN, III, Hamilton, OH (US); Chisomaga Ugochi NWACHUKWU, Cincinnati, OH (US); Katherine Ann STRASEMEIER, Cincinnati, OH (US); Greg Morgan VETTER, Hamilton, OH (US)

(21) Appl. No.: 16/125,820

(22) Filed: Sep. 10, 2018

Related U.S. Application Data
(60) Provisional application No. 62/563,688, filed on Sep. 27, 2017.

(51) Int. Cl. C11B 9/00; A61L 9/01; A61L 2/18

(52) U.S. Cl. CPC C11B 9/0034; C11B 9/0015; A61L 2/18; A61L 9/01; C11B 9/0061

(57) ABSTRACT

A freshening composition is disclosed. The freshening composition includes a sulfur-containing pro-perfume; at least 15 wt. % of a perfume mixture, based on the total weight of the freshening composition; and a carrier. Either the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; or the carrier comprises a propylene glycol; or the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof, and the carrier comprises a propylene glycol.

### FIELD

The present disclosure relates to stable freshening compositions and products comprising stable freshening compositions, and, more particularly, to freshening compositions including a sulfur containing pro-perfume.

### BACKGROUND

Perfume raw materials may be susceptible to degradation and environmental factors such as heat, light, and humidity can accelerate the degradation. The stability of a freshening composition may be dependent upon the perfume raw materials present in the freshening composition and the environmental impacts to the freshening composition. Degradation of perfume raw materials can cause color and or character changes. There is an ongoing need to minimize the degradation effect on perfume raw materials and freshening compositions having perfume raw materials.

### SUMMARY

### “Combinations:”

A. A freshening composition comprising:

a sulfur-containing pro-perfume;

at least 15 wt. % of a perfume mixture, based on the total weight of the freshening composition; and
a carrier, wherein either:
   (a) the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; or
   (b) the carrier comprises a propylene glycol; or
   (c) the perfume mixture comprises at least one perfume raw material selected from
the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof, and the carrier comprises a propylene glycol.
   B. The freshening composition of Paragraph A, wherein the sulfur-containing pro-perfume is a C4-C12 thio-damascone, more preferably the sulfur-containing pro-perfume is 3-(dodecylthio)-1-(2,6,6-trimethylcyclohex-3-en-1-yl)-1-butanone.
   C. The freshening composition of Paragraph A or B, wherein the carrier is a nonaqueous carrier.
   D. The freshening composition of any of Paragraphs A-C, wherein the carrier is present at up to 70 wt. %, more preferably up to 60 wt. %, most preferably up to 50 wt. %, based on the total weight of the freshening composition.
   E. The freshening composition of any of Paragraphs A-D, wherein the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; and wherein the carrier comprises a propylene glycol.
   F. The freshening composition of any of Paragraphs A-E, at least 30 wt. % of a perfume mixture, more preferably at least 40 wt. %, most preferably at least 50 wt. %.
   G. A freshening product comprising the freshening composition of any of Paragraphs A-F disposed in a transparent or translucent container.
   H. A freshening product comprising:

a freshening composition;

a reservoir for containing the freshening composition; and

a delivery engine in fluid communication with the freshening composition, wherein the delivery engine is selected from the group consisting of: wick, breathable membrane, gel, porous and semi-porous substrate, and combinations thereof,

wherein the composition comprises a sulfur-containing pro-perfume; a perfume mixture; a carrier, wherein either:
(a) the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; or
(b) the carrier comprises a propylene glycol; or
(c) the perfume mixture comprises at least one perfume raw material selected from
the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof, and the carrier comprises a propylene glycol.
   I. The freshening product of Paragraph H, wherein the sulfur-containing pro-perfume is a C4-C12 thio-damascone, more preferably the sulfur-containing pro-perfume is 3-(dodecylthio)-1-(2,6,6-trimethylcyclohex-3-en-1-yl)-1-butanone.
   J. The freshening product of Paragraph H or I, wherein the carrier is a nonaqueous carrier.
   K. The freshening product of any of Paragraphs H-J, wherein the carrier is present at up to 70 wt. %, more preferably up to 60 wt. %, most preferably up to 50 wt. %, based on the total weight of the freshening composition.
   L. The freshening product of any of Paragraphs H-K, at least 30 wt. % of a perfume mixture, more preferably at least 40 wt. %, most preferably at least 50 wt. %.
   M. The freshening product of any of Paragraphs H-L, wherein the reservoir is at least partially transparent or translucent.
   N. The freshening product of any of Paragraphs H-M further comprising an evaporative assistance element selected from the group consisting of: a heater, a fan, an agitator, and combinations thereof.
   O. The freshening product of any of Paragraphs H-N, wherein the perfume mixture further comprises one or more perfume raw materials selected from the group consisting of: 3-(1,3-Benzodioxol-5-yl)-2-methylpropanal, canthoxal, vanillin, ethyl vanillin, citral, ligustral, cinnamic aldehydes, and combinations thereof.

### BRIEF DESCRIPTION OF THE DRAWINGS

FIG. 1A is a schematic of an exemplary freshening product in the form of an electrical wall plug freshening product.

FIG. 1B is a perspective view of an exemplary cartridge of an freshening product having a microfluidic die and a wick that delivers the freshening composition to the microfluidic die.

FIG. 2 is a perspective view of an exemplary passive freshening product having a breathable membrane.

FIG. 3 is an exploded view of an exemplary passive freshening product having a breathable membrane.

FIG. 4 is a plot of the stability change of formulations with and without a sulfur-containing pro-perfume.

### DETAILED DESCRIPTION

The freshening compositions disclosed herein may provide increased stability, and, therefore, product life. The freshening composition include a sulfur-containing pro-perfume, perfume raw material(s), and one or more carriers.

As used herein, “freshening product” means products for treating or fragrancing the air or a surface including energized (i.e. electrically powered) freshening delivery systems including fan-based diffusers, liquid electric pluggable freshening products, electromechanical actuating diffusers; passive diffusers (i.e. not electrically powered) including membrane-based in-room freshening products, car vent freshening products

As used herein, “freshening composition” means a composition that includes one or more perfume raw materials that is intended to treat (e.g. eliminate or reduce/minimize malodors), fragrance, and/or freshen the air or a surface. The freshening composition may be used with or without an freshening product. Freshening compositions of the present invention include PRMs and may additionally include water, solubilizers, surfactants, diluents, malodor reducing actives, and perfume materials.

Sulfur-containing pro-perfumes have not been traditionally used in freshening products having a delivery engine in the form of a wick, membrane, and semi-porous substrate because the sulfur-containing pro-perfume may not evaporate through the delivery engine to provide the long-lasting freshness benefit known to be associated with such compounds. However, the sulfur-containing pro-perfume has been found to improve the stability in both aqueous and non-aqueous compositions for products with and without the use of a delivery engine in the form of a wick, membrane, and semi-porous substrate. The freshening composition may be single or multi-phase composition. The freshening composition may be aqueous, non-aqueous, or a multi-phase composition comprising aqueous and non-aqueous phases. Perfume raw materials and/or carriers may experience stability improvements from the incorporation of sulfur-containing pro-perfume in one or multiple phases of the freshening composition.

### Sulfur-Containing Pro-Perfume

The term “sulfur-containing pro-perfume” herein refers to a type of pro-perfume compound that contains sulfur. The term “pro-perfume” herein refers to compounds resulting from the reaction of perfume materials (“PRMs” or, singularly, “PRM”) with other chemicals, which have a covalent bond between one or more PRMs and these other chemicals. The PRM is converted into a new material called a pro-perfume compound, which then may release the original PRM (i.e., pre-converted) upon exposure to a trigger such as water or light or atmospheric oxygen. Suitable pro-perfume compounds and methods of making the same can be found in U.S. Pat. Nos. 7,018,978; 6,861,402; 6,544,945; 6,093,691; 6,165,953; and 6,096,918.

The sulfur-containing pro-perfume herein may comprise a compound of formula (I):

Y—S-G-Q (I)
wherein:
   (i) Y is a radical selected from the group consisting of (Y-1) to (Y-7) shown herein below, including isomeric forms:

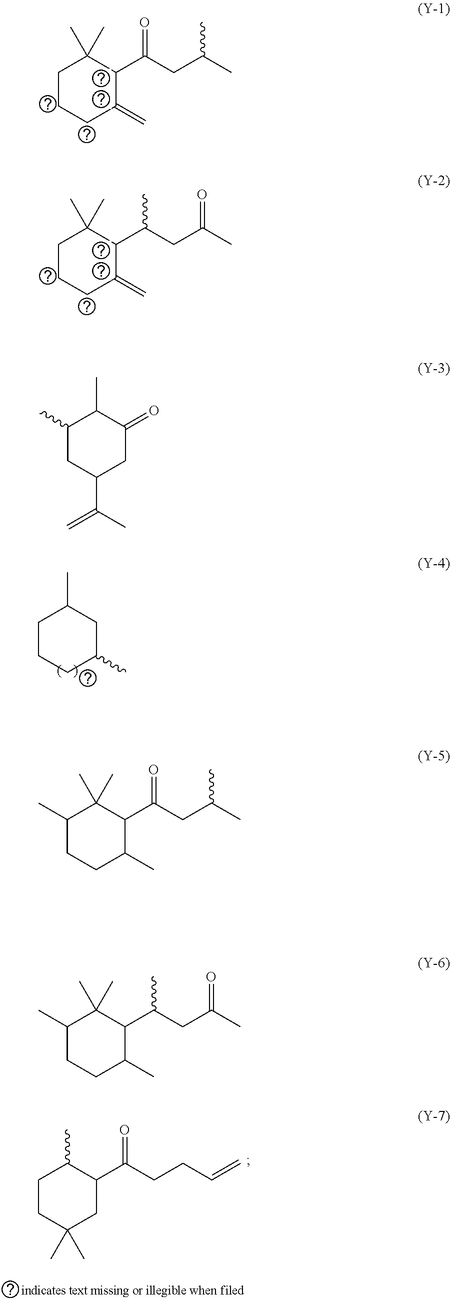

wherein the wavy lines represent the location of the sulfur (S) bond, and the dotted lines represent a single or double bond;
(ii) G is selected from a divalent or trivalent radical derived from a linear or branched alkyl or alkenyl radical having from 2 to 15 carbon atoms; and
(iii) Q is selected from a hydrogen, a —S—Y group, or a —NR²—Y group, wherein Y is independently selected as defined above, and R² is selected from a hydrogen or a C₁-C₃ alkyl group.

G may be a divalent or trivalent radical, preferably a divalent radical derived from a linear or branched alkyl or alkenyl radical having from 2 to 15 carbon atoms, substituted with one or more groups selected from the group consisting of —OR¹, —NR¹₂, —COOR¹, R¹ groups, and a combination thereof, wherein R¹ is selected from a hydrogen or a C₁ to C₆ alkyl or alkenyl group. Preferably, G is a divalent radical derived from a linear or branched alkyl or alkenyl radical having from 2 to 15 carbon atoms, substituted with at least one —COOR¹ group, preferably substituted with a —COOR¹ group, wherein R¹ is selected from a hydrogen or a C₁ to C₆ alkyl or alkenyl group. Even more preferably, G is a divalent radical derived from a linear alkyl radical having a —CH₂CH(COOR¹) group, wherein R¹ is a hydrogen or a methyl or ethyl group. G may be a divalent radical derived from a linear alkyl radical having from 8 to 15 carbon atoms which is either substituted or un-substituted.

The sulfur-containing pro-perfume may be a compound of formula (I) wherein Y is selected from Y-1, Y-2 or Y-3 groups as defined above, and G and Q are defined in any one of the above-described examples. The sulfur-containing pro-perfume may be a sulfide.

Preferably, the sulfur-containing pro-perfume is selected from the group consisting of methyl or ethyl 2-(4-oxo-4-(2,6,6-trimethylcyclohex-3-en-1-yl)butan-2-ylamino)-3-(4-oxo-4-(2,6,6-trimethylcyclohex-3-en-1-yl)butan-2-ylthio)propanate, methyl or ethyl 2-(4-oxo-4-(2,6,6-trimethylcyclohex-2-en-1-yl)butan-2-ylamino)-3-(4-oxo-4-(2,6,6-trimethylcyclohex-2-en-1-yl)butan-2-ylthio)propanate, methyl or ethyl 2-(2-oxo-4-(2,6,6-trimethylcyclohex-1-en-1-yl)butan-4-ylamino)-3-(2-oxo-4-(2,6,6-trimethylcyclohex-1-en-1-yl)butan-4-ylthio)propanate, methyl or ethyl 2-(2-oxo-4-(2,6,6-trimethylcyclohex-2-en-1-yl)butan-4-ylamino)-3-(2-oxo-4-(2,6,6-trimethylcyclohex-2-en-1-yl)butan-4-ylthio)propanate, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, and a combination thereof.

More preferably, the sulfur-containing pro-perfume compound is selected from the group consisting of 3-(dodecylthio)-1-(2,6,6-trimethylcyclohex-3-en-1-yl)-1-butanone, 4-(dodecylthio)-4-(2,6,6-trimethylcyclohex-2-enl-yl)-2-butanone, 4-(dodecylthio)-4-(2,6,6-trimethylcyclohex-1-en-1-yl)-2-butanone and 3-(dodecylthio)-5-isopropenyl-2-methylcyclohexanone, and a combination thereof. 3-(dodecylthio)-1-(2,6,6-trimethylcyclohex-3-en-1-yl)-1-butanone is the most preferred sulfur-containing pro-perfume compound, such as Haloscent® D available from Firmenich located in Geneva, Switzerland.

The sulfur-containing pro-perfume compound may be present at various levels in the composition. Preferably, the sulfur-containing pro-perfume compound is present in an amount from about 0.001 wt. % to about 5.0 wt. %, alternatively from about 0.001 wt. % to about 3.0 wt. %, alternatively from about 0.01 wt. % to about 1.0 wt. %, alternatively about 0.01 wt. % to about 0.5 wt. %, alternatively about 0.01 wt. % to about 0.1 wt. %, alternatively at least about 0.02%, alternatively at least about 0.02%, by weight of the freshening composition.

The freshening composition may comprise dodecyl thio-damascone having the general structure shown below.

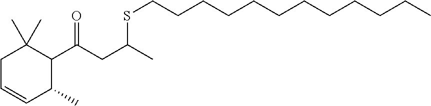 [SMILES: CCCCCCCCCCCCSC(C)CC(=O)C1[C@H](C)C=CCC1(C)C]

Thio-damascone may be present in an amount from about 0.01% to about 1.0%, alternatively from about 0.001 wt. % to about 5.0 wt. %, alternatively from about 0.001 wt. % to about 3.0 wt. %, alternatively from about 0.01 wt. % to about 1.0 wt. %, alternatively about 0.01 wt. % to about 0.5 wt. %, alternatively about 0.01 wt. % to about 0.1 wt. %, alternatively at least about 0.02%, alternatively at least about 0.02%, by weight of the freshening composition.

### Perfume Mixture

The freshening composition includes a perfume mixture comprising one or more perfume raw materials. Suitable perfume raw materials are disclosed in U.S. Pat. Nos. 5,663,134; 5,670,475; 5,783,544; 5,939,060; and 6,146,621.

The freshening composition may include various different PRMs. Exemplary PRMs are listed in TABLE 1 below.

TABLE 1
Perfume Raw Materials

| CAS No. | Name | CAS No. | Name |
|---|---|---|---|
| 31375-17-4 | 1-(P-Menthen-6(2)-Yl)-1- | 1195-79-5 | Fenchone |
| | Propanone | | |
| 68991-97-9 | 1,2,3,4,5,6,7,8-Octahydro-8,8- | 137-03-1 | Fleuramone |
| | Dimethyl-2-Naphthaldehyde | | |
| 1192-88-7 | 1-Cyclohexene-1-Carboxaldehyde | 71077-31-1 | Floral Super |
| 66327-54-6 | 1-Methyl-4-(4-Methylpentyl)-3- | 67634-14-4 | Floralozone |
| | Cyclohexenecarbaldehyde | | |
| 95962-14-4 | 2-(2-(4-Methyl-3-Cyclohexen-1- | 125109-85-5 | Florhydral |
| | Yl)Propyl)-Cyclopentanone | | |
| 74338-72-0 | 2,4,4,7-Tetramethyl-Oct-6-En-3- | | Formyl Tricyclodecan |
| | One | | |
| 1335-66-6 | 2,4,6-Trimethyl-3-Cyclohexene-1- | 14765-30-1 | Freskomenthe |
| | Carboxaldehyde | | |
| 25152-84-5 | 2,4-Decadienal | 10/1/6413 | Fructone |
| 68039-49-6 | 2,4-Dimethyl-3-Cyclohexen-1- | 68912-13-0 | Frutene |
| | Carbaldehyde | | |
| 68039-49-6 | 2,4-Dimethyl-3-Cyclohexene-1- | 706-14-9 | Gamma Decalactone |
| | Carboxaldehyde | | |
| 15764-16-6 | 2,4-Dimethylbenzaldehyde | 74568-05-1 | Gamma Undecalactone |
| 68737-61-1 | 2,4-Dimethylcyclohex-3-Ene-1- | 79-76-5 | Gamma-Ionone |
| | Carbaldehyde | | |
| 142-83-6 | 2,4-Hexadienal | 127-51-5 | Gamma-Methyl Ionone |
| 30361-28-5 | 2,4-Octadienal | 104-50-7 | Gamma-Octalactone |
| 24048-13-3 | 2,6,10-Trimethyl-5,9-Undecadien- | 108-29-2 | Gamma-Valero Lactone |
| | 1-A1 | | |
| 141-13-9 | 2,6,10-Trimethyl-9-Undecenal | 29214-60-6 | Gelsone |
| 116-26-7 | 2,6,6-Trimethyl-1,3-Diene | 5392-40-5 | Geranial |
| | Methanal | | |
| 472-66-2 | 2,6,6-Trimethyl-1-Cyclohexene-1- | 57934-97-1 | Givescone |
| | Acetaldehyde | | |
| 106-72-9 | 2,6-Dimethyl-5-Heptenal | 111-30-8 | Glutaraldehyde |
| 26370-28-5 | 2,6-Nonadienal | 111-30-8 | Glutaric Aldehyde |
| 103-95-7 | 2.Methyl-3(P-Isopropylphenyl)- | 34902-57-3 | Habanolide |
| | Propionaldehyde | | |
| 42370-07-0 | 2-Acetyl-3,3-Dimethyl-Norbornane | 24851-98-7 | Hedione |
| 112-54-9 | 2-Dodecanal | 1205-17-0 | Helional |
| 613-69-4 | 2-Ethoxybenzaldehyde | 120-57-0 | Heliotropin |
| 97-96-1 | 2-Ethylbutyraldehyde | 141773-73-1 | Helvetolide |
| 6728-26-3 | 2-Hexenal | 111-71-7 | Heptanal |
| 101-86-0 | 2-Hexyl 3-Phenyl Propenal | 79-78-7 | Hexalon |
| 90-02-8 | 2-Hydroxy Benzaldehyde | 66-25-1 | Hexenal |
| 35158-25-9 | 2-Isopropyl-5-Methyl-2-Hexenal | 101-86-0 | Hexyl Cinnamic Aldehyde |
| 101-39-3 | 2-Methyl 3-Phenyl Propenal | 7/7/2349 | Hexyl Iso-Butyrate |
| 96-17-3 | 2-Methyl Butyraldehyde | Specialty | Hs Raspberry |
| 19009-56-4 | 2-Methyl Deca-1-Al (2 Methyl | 90-87-9 | Hydrotropaldehyde |
| | Decanal) | | |
| 123-15-9 | 2-Methyl Valeraldehyde | 107-75-5 | Hydroxycitronellal |
| 110-41-8 | 2-Methyl-1-Undecanal | 120-72-9 | Indole |
| 623-36-9 | 2-Methyl-2-Pentenal | 1337-83-3 | Intreleven Aldehyde |
| 1205-17-0 | 2-Methyl-3-(3,4- | 14901-07-6 | Ionone Beta |
| | Methylenedioxyphenyl)Propanal | | |
| 41496-43-9 | 2-Methyl-3-Tolylproionaldehyde, | 1335-66-6 | Iso Cyclocitral |
| | 4-Dimethylbenzenepropanal (4- | | |
| | Dimethyl Benzenepropanal) | | |
| 80-54-6 | 2-Methyl-4-T- | 1335-66-6 | Iso Cyclocitral |
| | Butylphenyl)Propanal | | |
| 123-15-9 | 2-Methylpentanal | 95-41-0 | Iso Jasmone |
| 623-36-9 | 2-Methylpentenal | 659-70-1 | Iso-Amyl Iso-Valerate |
| 122-40-7 | 2-Pentyl-3-Phenylpropenoic | 78-84-2 | Isobutyraldehyde |
| | Aldehyde | | |
| 4411-89-6 | 2-Phenyl 2-Butenal | 54464-57-2 | Isocyclemone E |
| 93-53-8 | 2-Phenylproprionaldehyde | 1335-66-6 | Iso-Cyclo Citral |
| 125109-85-5 | 3-(3-Isopropyl-Phenyl)- | 70266-48-7 | Iso-Damascone |
| | Butyraldehyde | | |
| 103-95-7 | 3-(P-Isopropylphenyl)- | 54464-57-2 | Iso-E-Super |
| | Propionaldehyde | | |
| 4433-36-7 | 3,4,5,6-Tetrahydropseudoionone | 58430-94-7 | Iso-Nonyl Acetate |
| 139-85-5 | 3,4-Dihydroxybenzaldehyde | 590-86-3 | Isovaleraldehyde |
| 120-14-9 | 3,4-Dimethoxybenzaldehyde | 101-86-0 | Jasmonal H |
| 120-57-0 | 3,4-Methylene Dioxy | 41496-43-9 | Jasmorange |
| | Benzaldehyde | | |
| 134-96-3 | 3,5-Dimethoxy 4- | 2111-75-3 | L-4(1-Methylethenyl)-1- |
| | Hydroxybenzaldehyde | | Cyclohexene-1-Carboxaldehyde |
| 106-23-0 | 3,7-Dimethyl 6-Octenal | 112-54-9 | Lauric Aldehyde |
| 107-75-5 | 3,7-Dimethyl Octan-1-Al | 491-35-0 | Lepidine |
| 106-24-1 | 3,7-Dimethyl-2,6-Octadien-1-Al | 68039-49-6 | Ligustral |
| 7492-67-3 | 3,7-Dimethyl-6-Octenyl | 62518-65-4 | Lilestralis 33 |
| | Oxyacetaldehyde | | |
| 121-32-4 | 3-Ethoxy 4-Hydroxybenzaldehyde | 80-54-6 | Lilial |
| 590-86-3 | 3-Methyl Butyraldehyde | | Lime Aldehyde |
| 107-86-8 | 3-Methyl-2-Butenal | 78-70-6 | Linalool |
| 55066-49-4 | 3-Methyl-5-Phenyl Pentanal | 115-95-7 | Linalyl Acetate |
| 16630-52-7 | 3-Methylthiobutanal | 3720-16-9 | Livescone |
| 16251-77-7 | 3-Phenyl Butanal | 51414-25-6 | Lyral |
| 36306-87-3 | 4-(1-Ethoxyvinyl)-3,3,5,5,- | 80-54-6 | Lysmeral |
| | Tetramethyl-Cyclohexanone | | |
| 122-48-5 | 4-(4-Hydroxy-3-Methoxyphenyl)- | 67845-30-1 | Maceal |
| | 2-Butanone | | |
| 31906-04-4 | 4-(4-Hydroxy-4-Methyl Pentyl)-3- | 20407-84-5 | Mandarinal |
| | Cyclohexene-1-Carboxaldehyde | | |
| 4927-36-0 | 4-Damascol | 20407-84-5 | Mandarine Aldehyde |
| 10031-82-0 | 4-Ethoxybenzaldehyde | 39255-32-8 | Manzanate |
| 4748-78-1 | 4-Ethyl Benzaldehyde | 62518-65-4 | Mefloral |
| 122-03-2 | 4-Isopropyl Benzaldehyde | 55066-49-4 | Mefranal |
| 621-59-0 | 4-Methoxy 3-Hydroxy | 68991-97-9 | Melafleur |
| | Benzaldehyde | | |
| 5703-26-4 | 4-Methylphenylacetaldehyde | 106-72-9 | Melonal |
| 18127-01-0 | 4-T-Butylbenzenepropionaldehyde | 30772-79-3 | Melozone |
| 80-54-6 | 4-Tert-Butyl-Alpha-Methyl- | 89-80-5 | Menthone |
| | Hydrocinnamaldehyde | | |
| 32210-23-4 | 4-Tertiary Butyl Cyclohexyl | 62439-41-2 | Methoxy Melonal |
| | Acetate | | |
| | 4-Tricyclo5210-2,6decylidene- | 1504-74-1 | Methoxycinnamaldehyde (Ortho) |
| | 8butanal | | |
| 37609-25-9 | 5-Cyclohexadecenone | 24851-98-7 | Methy-Dihydrojasmonate |
| 33704-61-9 | 6,7-Dihydro-1,1,2,3,3-Pentamethyl- | 93-08-3 | Methyl Beta Naphthyl Ketone |
| | 4(5h)-Indanone | | |
| | 6-Isopropyldecahydro-2-Naphtone | 32388-55-9 | Methyl Cedrylone Major |
| 62439-41-2 | 6-Methoxy-2,6-Dimethylheptanal | 103-26-4 | Methyl Cinnamate |
| 107-75-5 | 7-Hydroxy-3,7-Dimethyl Octan-1- | 68480-14-8 | Methyl Cyclocitrone |
| | Al | | |
| 123-69-3 | 8-Hexadecenolide | 24851-98-7 | Methyl Dihydro Jasmonate |
| 84697-09-6 | Acalea | 93-16-3 | Methyl Isoeugenol |
| 75-07-0 | Acetaldehyde | 110-41-8 | Methyl Nonyl Acetaldehyde |
| 98-86-2 | Acetophenone | 112-12-9 | Methyl Nonyl Ketone |
| 141-13-9 | Adoxal | 19009-56-4 | Methyl Octyl Acetylaldehyde |
| 19009-56-4 | Aldehyde C-11 MOA | 93-92-5 | Methyl Phenyl Carbinyl Acetate |
| 110-41-8 | Aldehyde C12 MNA | 119-36-8 | Methyl Salicylate |
| 123-68-2 | Allyl Caproate | 122-00-9 | Methyl-Acetophenone |
| 122-40-7 | Alpha-Amylcinnamic Aldehyde | 93-08-3 | Methyl-Beta-Naphthyl-Ketone |
| 6753-98-6 | Alpha-Caryophyllene | 96-17-3 | Methylbutyraldehyde |
| 43052-87-5 | Alpha-Damascone | 32388-55-9 | Methyl-Cedrenyl-Ketone |
| 101-86-0 | Alpha-Hexylcinnamaldehyde | 32388-55-9 | Methyl-Cedrylone |
| 127-41-3 | Alpha-Ionone | 101-39-3 | Methylcinnamaldehyde |
| 101-39-3 | Alpha-Methyl Cinnamic Aldehyde | 110-93-0 | Methyl-Heptenone |
| 127-42-4 | Alpha-Methyl Ionone | 67633-95-8 | Methyl-Lavender-Ketone |
| 101-39-3 | Alpha-Methylcinnamaldehyde | 7492-67-3 | Muget Aldehyde 50 |
| 103-95-7 | Alpha-Methyl-P-Isopropyl Phenyl | 541-91-3 | Muscone |
| | Propyl Aldehyde | | |
| 101-86-0 | Alpha-N-Hexyl-Cinnamaldehyde | 33704-61-9 | Musk Indanone |
| 80-56-8 | Alpha-Pinene | 21145-77-7 | Musk Plus |
| 628-63-7 | Amyl- Acetate | 37677-14-8 | Myrac Aldehyde |
| 122-40-7 | Amyl Cinnamic Aldehyde | 564-94-3 | Myrtenal |
| 495-85-2 | Amylaldehyde | | |
| 123-11-5 | Anisaldehyde | 127-43-5 | N-Beta-Methyl Ionone Isomer |
| 123-11-5 | Anisic Aldehyde | 173445-65-3 | Neo Hivernal |
| 6/6/5462 | Anisylpropanal | 56973-85-4 | Neobutenone |
| 100-52-7 | Benzaldehyde | 106-26-3 | Neral |
| 104-53-0 | Benzenepropanal | 124-19-6 | Nonanal |
| 119-61-9 | Benzophenone | 18829-56-6 | Nonenal |
| 140-11-4 | Benzyl Acetate | 86803-90-9 | Octahydro-5-Methoxy-4,7- |
| | | | Methano-1H-Indene-2- |
| | | | Carboxaldehyde |
| 100-51-6 | Benzyl Alcohol | 124-13-0 | Octanal |
| 120-51-4 | Benzyl Benzoate | 2548-87-0 | Octenal |
| 118-58-1 | Benzyl Salicylate | 54082-68-7 | Onicidal (Muguet Undecadienal) |
| 2550-26-7 | Benzyl-Acetone | 8028-48-6 | Orange Oil Tarocco |
| Specialty | Berry Wescorps | 16587-71-6 | Orivone |
| 65885-41-8 | Beta Methyl Benzenepropanal | 59323-76-1 | Oxane |
| 432-25-7 | Beta-Cyclocitral | 80-54-6 | P.T. Bucinal |
| 35044-68-9 | Beta-Damascone | 5471-51-2 | Para Hydroxy Phenyl Butanone |
| 928-96-1 | Beta-Gamma Hexanol | 67634-14-4 | Para-Ethyl-Alpha,Alpha- |
| | | | Dimethyl Hydrocinnamaldehyde |
| 14901-07-6 | Beta-Ionone | 100-06-1 | Para-Methoxy-Acetophenone |
| 128-37-0 | BHT | 98-53-3 | Para-Tert-Butyl-Cyclohexanone |
| 18127-01-0 | Bourgeonal | 106-02-5 | Pentadecanolide |
| 75147-23-8 | Buccoxime | 110-62-3 | Pentanal |
| 123-72-8 | Butyraldehyde | 111-30-8 | Pentanedial |
| 76-22-2 | Camphor | 2111-75-3 | Perillaldehyde |
| 6/6/5462 | Canthoxal | 103-60-6 | Phenoxy Ethyl Iso-Butyrate |
| 99-49-0 | Carvone | 101-48-4 | Phenyl Acetaldehyde Dimethyl |
| | | | Acetal |
| 55418-52-5 | Cassione (Heliotropin Acetone) | 4411-89-6 | Phenyl Butenal |
| Specialty | Cassis Base | 60-12-8 | Phenyl Ethyl Alcohol |
| 139-85-5 | Catechaldehyde | 103-48-0 | Phenyl Ethyl Iso-Butyrate |
| 3720-16-9 | Celery Ketone | 14371-10-9 | Phenyl Propenal, 3-Phenyl-2- |
| | | | Propenal |
| 104-55-2 | Cinnamic Aldehyde | 122-97-4 | Phenyl Propyl Alcohol |
| 103-54-8 | Cinnamyl Acetate | 122-78-1 | Phenylacetaldehyde |
| 6728-31-0 | Cis Heptenal | 564-94-3 | Pin-2-Ene-1-Carbaldehyde |
| 488-10-8 | Cis-Jasmone | 33885-51-7 | Pino Acetaldehyde |
| 5392-40-5 | Citral | 41724-19-0 | Plicatone |
| 106-23-0 | Citronellal | 123-11-5 | P-Methoxybenzene Aldehyde |
| 107-75-5 | Citronellal Hydrate | 101-39-3 | P-Methyl-Alpha- |
| | | | Pentylcinnamaldehyde |
| 106-22-9 | Citronellol | 107898-54-4 | Polysantol |
| 7492-67-3 | Citronellyl Oxyacetaldehyde | 52474-60-9 | Precyclemeone B |
| 120-14-9 | Corps 4322 (Vanillin Methyl Ether) | 1191-16-8 | Prenyl Acetate |
| Specialty | Corps Iris | 123-38-6 | Propanal |
| 91-64-5 | Coumarin | 123-38-6 | Propionaldehyde |
| 122-03-2 | Cuminaldehyde | 90105-92-3 | Prunella |
| 68039-49-6 | Cyclal C | 104-09-6 | P-Tolylacetaldehyde |
| 103-95-7 | Cyclamen Aldehyde | 78-98-8 | Pyruvaldehyde |
| 7775-00-0 | Cyclemax | 82461-14-1 | Rhubafuran |
| 68738-96-5 | Cyclemone A | 116-26-7 | Safranal |
| 91462-24-7 | Cyclic Ethylene Dodecanedioate | 90-02-8 | Salicylaldehyde |
| 31906-04-4 | Cyclohexenyl-Carboxaldehyde | 41496-43-9 | Satinaldehyde |
| 502-72-7 | Cyclopentadecanone | 86803-90-9 | Scentenal |
| 103-95-7 | Cyclosal | 104-09-6 | Syringaldehyde |
| 103-95-7 | Cymal | 21944-98-9 | Tangerinal |
| 43052-87-5 | Damarose Alpha | 1322-58-3 | Tetrameran |
| 23696-85-7 | Damascenone | 22471-55-2 | Thesaron |
| 35044-68-9 | Damascone Beta | 21145-77-7 | Tonalid |
| 112-31-2 | Decanal | 18829-55-5 | Trans Heptenal |
| 4819-67-4 | Delphone | 24680-50-0 | Trans-4- |
| | | | Methoxycinnamaldehyde |
| 57378-68-4 | Delta-Damascone | 30168-23-1 | Tricyclodecylidenebutanal |
| 18479-58-8 | Dihydro Myrcenol | 10486-19-8 | Tridecanal |
| 17283-81-7 | Dihydro-Beta-Ionone | 16251-77-7 | Trifernal |
| 5988-91-0 | Dihydrocitronellal | 68039-49-6 | Triplal |
| 1128-08-1 | Dihydrojasmone | 67801-65-4 | Triplal Extra |
| 85-91-6 | Dimethyl Anthranilate | 27939-60-2 | Trivertal |
| 151-05-3 | Dimethyl Benzyl Carbinyl Acetate | 11245-8 | Undec-10-En-1-Al (10- |
| | | | Undecenal) |
| 10094-34-5 | Dimethyl Benzyl Carbinyl Butyrate | 104-67-6 | Undecalactone |
| 2550-11-0 | Dimethyl-Octenone | 81782-77-6 | Undecavertol |
| 5989-27-5 | D-Limonene | 112-44-7 | Undecanal |
| 34590-94-8 | Dowanol DPM Isomer | 110-62-3 | Valeraldehyde |
| 55418-52-5 | Dulcinyl | 121-33-5 | Vanillin |
| 30168-23-1 | Duplical | 20665-85-4 | Vanillin Isobutyrate |
| 75-07-0 | Ethanal | 65443-14-3 | Veloutone |
| | Eth-Me-Ph Glycidate Isomer | 120-14-9 | Veratraldehyde |
| 39255-32-8 | Ethyl 2 Methyl Pentanoate | 1728-46-7 | Verdone |
| 11/8/4940 | Ethyl Maltol | 88-41-5 | Verdox |
| 35044-59-8 | Ethyl Safranate | 88-41-5 | Verdox Major |
| 121-32-4 | Ethyl Vanillin | 66327-54-6 | Vernaldehyde |
| 7452-79-1 | Ethyl-2-Methyl Butyrate | 32210-23-4 | Vertenex |
| 105-95-3 | Ethylene Brassylate | 68039-49-6 | Vertocitral |
| 470-82-6 | Eucalyptol | 1335-46-2 | Xandralia (Methyl) |
| 97-53-0 | Eugenol | 472-66-2 | B-Homocyclocitral |
| 93-28-7 | Eugenyl Acetate | | |

The perfume mixture may comprise one or more perfume raw materials selected from the group consisting of: dihydro myrcenol; dimethyl benzyl carbinyl acetate; ethyl vanillin; florhydral; nonanal; undecanal; vanillin; beta gamma hexanol; decanal; citronellol; and combinations thereof.

The perfume mixture may also include one or more perfumer raw materials selected from the group consisting of: 3-(1,3-Benzodioxol-5-yl)-2-methylpropanal, canthoxal, vanillin, ethyl vanillin, citral, ligustral, cinnamic aldehydes, and combinations thereof.

The freshening composition may comprise from greater than 10 wt. %, alternatively greater than 15 wt. %, alternatively greater than 20 wt. %, alternatively greater than 30 wt. %, alternatively greater than 40 wt. %, alternatively greater than 50 wt. %, alternatively greater than 60 wt. %, alternatively greater than 70 wt. %, alternatively greater than 85 wt. %, of a perfume mixture, alternatively about 10 wt. % to about 90 wt. %, alternatively about 20 wt. % to about 90 wt. %, alternatively about 30 wt. % to about 90 wt. %, based on the total weight of the freshening composition.

The weight ratio of perfume mixture to sulfur-containing pro-perfume may be about 6:1 to about 50:1, or about 6:1 to about 35:1, or about 8:1 to about 25:1, or about 10:1 to about 20:1, by weight of the composition.

The weight ratio of perfume mixture to thio-damascone may be about 6:1 to about 50:1, or about 6:1 to about 35:1, or about 8:1 to about 25:1, or about 10:1 to about 20:1, by weight of the composition.

### Carrier

The sulfur-containing pro-perfume has been found to improve the stability of both aqueous and non-aqueous freshening compositions. The freshening composition includes one or more carriers. The carrier may be aqueous or non-aqueous. The carrier may be selected from the group consisting of: a solvent and/or a diluent.

The carrier may be present in the freshening composition at a level of up to and including 80 wt. %, alternatively up to and including 70 wt. %, alternatively up to and including 60 wt. %, alternatively up to and including 50 wt. %, alternatively up to and including 40 wt. %, alternatively up to and including 30 wt. %, alternatively up to and including 20 wt. %, alternatively up to and including 15 wt. %, alternatively up to and including 10 wt. %, by total weight of the freshening composition.

The carrier may include a solvent, diluent, or combinations thereof. The solvent or diluent may be a glycol selected from the group consisting of: propylene glycol, dipropylnene glycol, tripropylene glycol. The solvent or diluent may be selected from the group consisting of: dipropylene glycol methyl ether (“DPM”), tripropylene glycol methyl ether (“TPM”), 3-methoxy-3-methyl-1-butanol (“MMB”), volatile silicone oil, and dipropylene glycol esters of methyl, ethyl, propyl, butyl, ethylene glycol methyl ether, ethylene glycol ethyl ether, diethylene glycol methyl ether, diethylene glycol ethyl ether, isopropyl myristate, or any VOC under the tradename of Dowanol™ glycol ether, and combinations thereof.

Some carriers may also experience stability improvements from the addition of a sulfur-containing pro-perfume. For example, carriers such as propylene glycols, including mono, di, or tri-propylene glycols may exhibit stability enhancement when combined with sulfur-containing pro-perfume.

The carrier may include water.

### Active Agents

The freshening composition may include an active agent. Active agents provide cleaning, surface care protection, fabric conditioning or softening, fabric refreshing, de-wrinkling, air freshening, air deodorizing, malodor removal, skin moisturizing, body deodorizing, or like benefits. An active agent does not include water or deionized water.

In a freshening composition, the active agents may deliver a genuine malodor removal benefit. A genuine malodor removal benefit is defined as both a sensory and analytically measurable (such as by GC) malodor reduction. Thus, if the freshening composition delivers a genuine malodor removal benefit, the freshening composition will not function merely by using perfume to cover up or mask odors. If the freshening product is provided with a malodor controlling agent, the freshening product may utilize one or more of several types of odor control mechanisms. One suitable malodor controlling agent is cyclodextrin.

Active agents might also include surfactants, emulsifiers, solubilizers, polymers, malodor counteractants such as cyclodextrin, hydrogen peroxide, buffers, zinc ions, etc.

### Freshening Product

The freshening composition may be used with an freshening product to deliver the perfume mixture to the atmosphere and/or a surface. It is contemplated that the freshening product may be configured for use in a variety of applications to deliver the perfume mixture to the atmosphere and/or a surface.

For example, the freshening product may be configured as an energized device. An exemplary energized device may be an electrical device. The energized device may be an electrical wall plug or battery operated freshening device having a delivery engine, such as a wick, that is used to transport a freshening composition and/or evaporate a freshening composition therefrom; or other heating devices (e.g. devices powered by chemical reactions such as catalyst fuel systems; solar powered devices, etc.). In such devices, the delivery engine is designed to transport a freshening composition and/or evaporate a freshening composition therefrom. The energized device may also include a microfluidic die having either a heater(s) or piezo crystal(s) that are used to dispense droplets of the freshening composition into the air. An exemplary microfluidic, energized device is described in U.S. Patent Application No. 62/483,496, entitled “MICROFLUIDIC DELIVERY DEVICE AND METHOD FOR DISPENSING A FLUID COMPOSITION UPWARD INTO THE AIR”.

When the delivery engine is used to evaporate the freshening composition therefrom, the delivery engine may be placed next to one or more evaporative assistance elements, such as a heater, to disperse the freshening composition in the atmosphere.

The delivery engine may be configured in various ways. For example, the delivery engine may be in the form of a wick, membrane, gel, porous or semi-porous substrate, including a felt pad.

If the freshening product includes a delivery engine in the form of a wick, the wick may be configured to have various different shapes and sizes. For example, the wick may have a cylindrical or an elongate cube shape. The wick may be defined by a length and a diameter or width, depending on the shape. The wick may have various lengths. For example, the length of the wick may be in the range of about 1 millimeter (“mm”) to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, or at least 4 mm

A wick may exhibit a density. The wick density may be in the range of about 0.100 grams/cm³ (“g/cc”) to about 1.0 g/cc.

A wick may comprise a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as PE, HDPE or other polyolefins. For example, the wick may be made from a plastic material such as polyethylene or a polyethylene blend.

Instead of evaporating the freshening composition from the delivery engine, the delivery engine may transport the freshening composition to a microfluidic die or an evaporative surface. For example, the delivery engine may transport the fluid composition, through capillary action, to a microfluidic die that uses a heater or piezo crystal to atomize or disperse droplets of the freshening composition into the atmosphere.

The evaporative surface may be integral or separate from the evaporative assistance element and/or the delivery engine. The evaporative surface may be configured as a porous or semi-porous substrate, a bowl or plate, including a plastic, glass, or metal bowl or plate, and combinations thereof.

When an evaporative assistance element is used, the evaporative assistance element may be configured in various ways. The evaporative assistance element may be used to achieve the evaporation of a freshening composition from an freshening product. For example, the evaporative assistance element may be selected from the group consisting of a heater, a fan, an agitation member or agitator, both powered agitator and manual agitator, or combinations thereof. The evaporative assistance element may also include a heating element to heat the liquid volatile composition, a chemical constituent to speed evaporation or release rates, use of a chemically heated membrane to provide increased evaporation via exothermic reaction, or synergistic combinations thereof.

An energized device having an evaporative assistance element in the form of a heater may be configured to heat the delivery engine to various temperatures. For example, the energized device may be configured such that the heater heats the evaporative surface, such as a wick, membrane, gel, porous or semi-porous substrate such as a felt pad, to a temperature of about 30° C. to about 150° C. An energized device may include a control system such that the heater temperature is adjustable. The control system may also cycle the heater temperature to have greater control over the evaporation of the freshening composition.

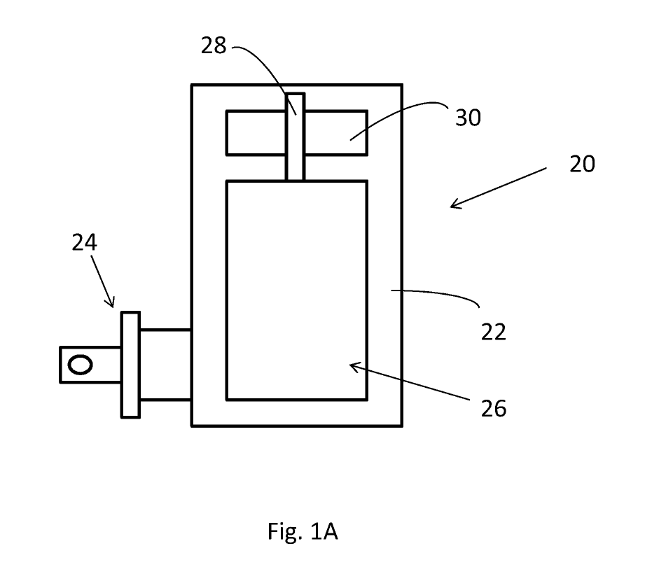

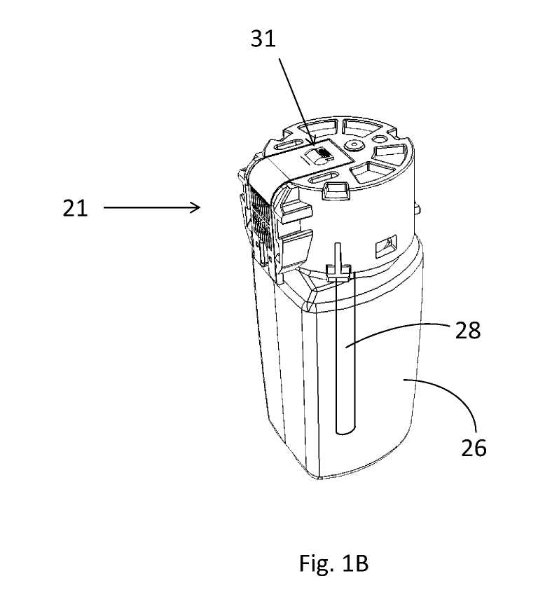

An exemplary energized device is shown in FIG. 1A in the form of an electrical wall plug freshening product 20. The wall plug freshening product 20 may include a housing 22, and the housing 22 is supported on an electrical outlet by a plug 24 that is at least indirectly joined to the housing 22. The freshening product 20 further comprises at least one reservoir 26 for containing the freshening composition. The housing 22 may serve as a holder for the reservoir(s) and any of the other components of the freshening product. The freshening product comprises a delivery engine in the form of a wick 28 and an evaporative assistance element in the form of a heater 30 for dispensing the volatile material. While FIG. 1A illustrates one reservoir, one evaporative assistance element, and one delivery engine, it is to be appreciated that the freshening product may include more than one reservoir, evaporative assistance element, and/or delivery engine. If the freshening product includes more than one reservoir, each reservoir may contain a different freshening composition or may contain the same freshening composition.

FIG. 1B illustrates a cartridge 21 of an exemplary freshening product comprising a microfluidic die. A cartridge 21 comprising a microfluidic die, such as shown in FIG. 1B, may include a reservoir 26 for containing the freshening composition, a delivery engine in the work of a wick 28 that is in fluid communication with the reservoir 26 and the freshening composition contained with the reservoir 26, and a microfluidic die 31. The microfluidic die 31 may include a heater(s) or piezo crystal(s) that is used to atomization the freshening composition to dispense the freshening composition into the atmosphere. The cartridge may be connected with a housing that supplies electricity to the microfluidic die 31.

The freshening product may also be configured as a passive diffuser apparatus that includes a breathable membrane for diffusing freshening composition.

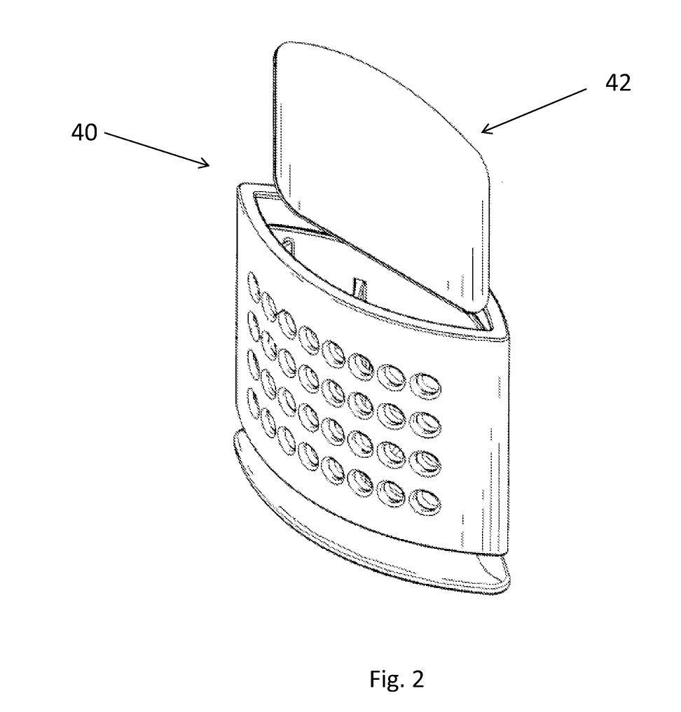

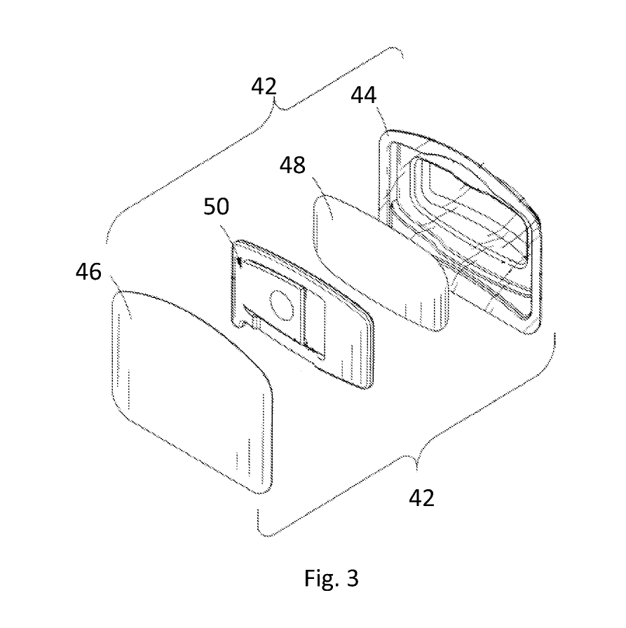

For example, as shown in FIGS. 2 and 3, the apparatus 40 for delivering a freshening composition may comprise a delivery engine 42 having a liquid reservoir 44 for containing a freshening composition and a breathable membrane 46 enclosing the liquid reservoir 44, such as disclosed in U.S. Pat. Nos. 8,709,337 and 8,931,711. A breathable membrane 46 is a vapor permeable membrane that prevents free flow of liquid out of the membrane, thus addressing leakage problems. Suitable membranes include, but are not limited to, UHMWPE-type membrane optionally filled with silica as described in U.S. Pat. No. 7,498,369. Such UHMWPE membranes include Daramic™ V5, available from Daramic, Solupor®, available from DSM (Netherlands), and Teslin™ SP1100HD, available from PPG Industries, and combinations thereof. Other suitable breathable membranes include any permeable polymeric, thermoplastic, or thermoset material, including acetal, acrylic, cellulosic, fluoroplastic, polyamide, polyester, polyvinyl, polyolefin, styrenic, etc, alone, co-extruded, woven or non-woven, mixed or in combination with elastomers, rubber, solids, silicas, or combinations thereof. Also suitable are Hytrel™ available from Dupont or Lotryl™ available from Arkema. The delivery engine 42, such as shown in FIG. 3, may also include a rupturable substrate 48 that seals the freshening composition in the liquid reservoir until a rupture mechanism 50 is engaged to when the apparatus is to be used by the consumer. When the consumer is ready to use the apparatus, the consumer can rupture the rupturable substrate 48 with the rupture mechanism 50, which allows the freshening composition in the liquid reservoir 44 to contact the breathable membrane.

The freshening composition can be packaged in any suitable package to form a freshening product. The package may be in the form of a spray dispenser. One suitable spray dispenser is a plastic aerosol dispenser. The dispenser may be constructed of polyethylene such as a high density polyethylene; polypropylene; polyethyleneterephthalate (“PET”); vinyl acetate, rubber elastomer, and combinations thereof. The spray dispenser may be made of clear PET. Another suitable spray dispenser includes a continuous action sprayer, such as FLAIROSOL™ dispenser from Afa Dispensing Group. The FLAIROSOL™ dispenser includes a bag-in-bag or bag-in-can container with a pre-compression spray engine, and aerosol-like pressurization of the freshening composition.

A pressurized spray dispenser may include a propellant. Various propellants may be used. The propellant may comprise hydrocarbon(s); compressed gas(es), such as nitrogen, carbon dioxide, air; liquefied gas(es) or hydrofluoro olefin (“HFO”); and mixtures thereof.

### Examples

Perfume raw materials or perfume mixtures were evaluated with and without a sulfur-containing pro-perfume present for stability analysis. The example formulations including a sulfur-containing pro-perfume are shown in Table 2. The sulfur-containing pro-perfume is 3-(dodecylthio)-1-(2,6,6-trimethylcyclohex-3-en-1-yl)-1-butanone, sold under the tradename Haloscent® D by Firmenich. The perfume mixture named “Perfume Oriental Retreat #1” without the addition of Haloscent® D is one of the perfume mixtures used in the AMBI PUR™ 3VOLUTION™ air freshener device sold in market today.

TABLE 2
Example Formulations

| | PRM | Haloscent ® D |
|---|---|---|
| PRM Formulation | Wt % | Wt % |
| Dihydro Myrcenol | 99.970% | 0.0300% |
| Dimethyl Benzyl Carbinyl Acetate | 99.970% | 0.0300% |
| Ethyl Vanillin (10% in DPG) | 99.970% | 0.0300% |
| Florhydral | 99.970% | 0.0300% |
| Hexal Cinnamic Aldehyde | 99.970% | 0.0300% |
| Lauric Aldehyde | 99.970% | 0.0300% |
| Linalool | 99.970% | 0.0300% |
| Nonanal | 99.970% | 0.0300% |
| Octyl Aldehyde | 99.970% | 0.0300% |
| Octyl Alcohol | 99.970% | 0.0300% |
| Undecanal | 99.970% | 0.0300% |
| Vanillin (10% in Dipropylene Glycol) | 99.970% | 0.0300% |
| Perfume Oriental Retreat #1 | 99.970% | 0.0300% |
| FP Oriental Retreat #1 | 99.970% | 0.0300% |
| Beta Gamma Hexanol | 99.970% | 0.0300% |
| Decanal | 99.970% | 0.0300% |
| Dipropylene Glycol | 99.970% | 0.0300% |

The initial color of each composition was first measured and then samples are stored in a sealed glass jar at 50 degrees Celsius for 21 days to simulate accelerated aging. After aging for 21 days, color measurements were taken again. Equivalent Stability for Ambient Conditions can predicted based on Arrhenius Equation and Industry Standard Models such as the ICH Models (International Council for Harmonization of Technical Requirements) per given temperature. In general, 2 weeks at 50° C. has been found to be able to predict about 1 year of aging at ambient temperature; 1 month at 50° C. has been found to be able to predict about 2 years of aging at ambient temperature.

The color of each composition is quantitatively measured via a HunterLab LabScan XE spectrophotometer according to the manufacturers published instruction manual to measure the L*a*b* values. The HunterLab LabScan XE spectrophotometer gives Hunter L*-a*-b*color space readings for each sample. The Hunter L*-a*-b* color space is organized in a cube form. The L* axis runs from top to bottom. The maximum value for L* is 100 and the minimum value is zero, which would be black. The a* and b* axes have no specific numerical limits. A positive a* value is red, while a negative a* value is green. For these experiments, we primarily focus on the Hunter b* value, which is a measure of how yellow or blue a sample is. A positive b* value is yellow, while a negative b* value is blue. The more positive a b* value is, the more “yellow” a sample is. Stated another way, as the b* value increases, the darker the color of a sample is (orange, brown, red, etc.). Conversely, the more negative the b* value is, the bluer the sample is (clear, white, blue).

For these experiments, we also compare the Initial b* color values to Aged b* values after 21 days for notable change in color. The Aged b* value is the measurement taken after 21 days of aging at 50° C. The bigger the difference from Initial b* to Aged b*, the more unstable the sample is, and conversely, the smaller the difference from Initial b* to Aged b*, the more stable the product is.

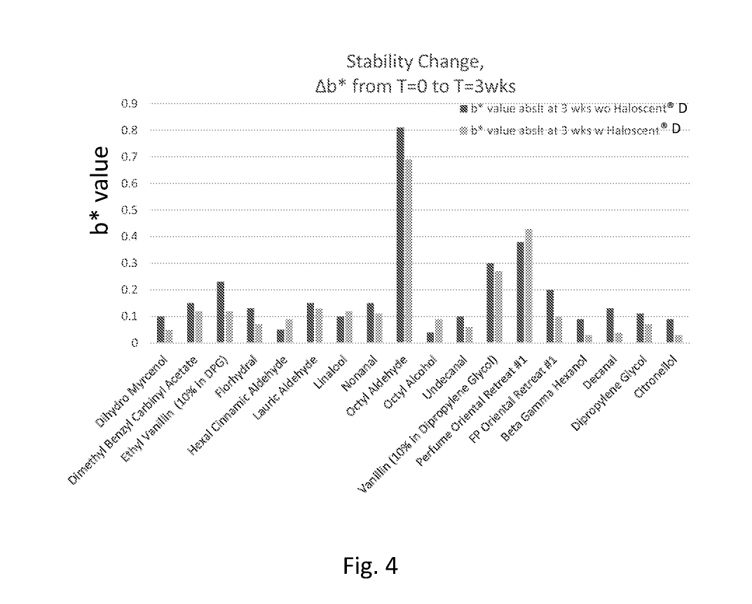

As shown in FIG. 4, combining the perfume raw materials dihydro myrcenol, dimethyl benzyl carbinyl acetate, ethyl vanillin, florhydral, nonanal, undecanal, vanillin, beta gamma hexanol, decanal, and citronellol with a sulfur-containing pro-perfume show stability improvement in Aged b* values over the same materials in the absence of a sulfur-containing pro-perfume. As shown in FIG. 4, dipropylene glycol, a propylene glycol carrier, also shows stability improvement in the presence of a sulfur-containing pro-perfume. As shown in FIG. 4, it was found that only select perfume raw material show stability improvement from the incorporation of a sulfur-containing pro-perfume.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A freshening composition comprising:
a sulfur-containing pro-perfume;
at least 15 wt. % of a perfume mixture, based on the total weight of the freshening composition; and
a carrier, wherein either:
(a) the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; or
(b) the carrier comprises a propylene glycol; or
(c) the perfume mixture comprises at least one perfume raw material selected from
the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof, and the carrier comprises a propylene glycol.

2. The freshening composition of claim 1, wherein the sulfur-containing pro-perfume is a C4-C12 thio-damascone.

3. The freshening composition of claim 1, wherein the carrier is a nonaqueous carrier.

4. The freshening composition of claim 1, wherein the carrier is present at up to 70 wt. %, based on the total weight of the freshening composition.

5. The freshening composition of claim 1, wherein the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; and wherein the carrier comprises a propylene glycol.

6. The freshening composition of claim 1, at least 30 wt. % of a perfume mixture.

7. A freshening product comprising the freshening composition of claim 1 disposed in a transparent or translucent container.

8. A freshening product comprising:
a freshening composition;
a reservoir for containing the freshening composition; and
a delivery engine in fluid communication with the freshening composition, wherein the delivery engine is selected from the group consisting of: wick, breathable membrane, gel, porous and semi-porous substrate, and combinations thereof,
wherein the composition comprises a sulfur-containing pro-perfume; a perfume mixture; a carrier, wherein either:
(a) the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; or
(b) the carrier comprises a propylene glycol; or
(c) the perfume mixture comprises at least one perfume raw material selected from
the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof, and the carrier comprises a propylene glycol.

9. The freshening product of claim 8, wherein the sulfur-containing pro-perfume is a C4-C12 thio-damascone.

10. The freshening product of claim 8, wherein the carrier is a nonaqueous carrier.

11. The freshening product of claim 8, wherein the carrier is present at up to 50 wt. %, based on the total weight of the freshening composition.

12. The freshening product of claim 8, wherein the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; and wherein the carrier comprises a propylene glycol.

13. The freshening product of claim 8, at least 40 wt. % of a perfume mixture.

14. The freshening product of claim 8, wherein the reservoir is at least partially transparent or translucent.

15. The freshening product of claim 8 further comprising an evaporative assistance element selected from the group consisting of: a heater, a fan, an agitator, and combinations thereof.

16. The freshening product of claim 8, wherein the freshening composition further comprising an active agent.

17. The freshening product of claim 8, wherein the perfume mixture further comprises one or more perfume raw materials selected from the group consisting of: 3-(1,3-Benzodioxol-5-yl)-2-methylpropanal, canthoxal, vanillin, ethyl vanillin, citral, ligustral, cinnamic aldehydes, and combinations thereof.

18. A freshening composition comprising:
about 0.02 wt. % to about 1.0 wt. %, based on the weight of the composition, of a sulfur-containing pro-perfume;
about 0.2 wt. % to about 1.4 wt. %, based on the weight of the composition, of a perfume mixture; and
a carrier,
wherein either:
(a) the perfume mixture comprises at least one perfume raw material selected from the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof; or
(b) the carrier comprises a propylene glycol; or
(c) the perfume mixture comprises at least one perfume raw material selected from
the group consisting of: dihydro myrcenol, dimethyl benzyl carbinyl acetate, ethyl vanillin, florhydral, nonanal, undecanal, vanillin, beta gamma hexanol, decanal, citronellol, and combinations thereof, and the carrier comprises a propylene glycol.

19. The freshening composition of claim 18, wherein the carrier is an aqueous carrier and wherein the sulfur-containing pro-perfume is a C4-C12 thio-damascone.

20. A freshening product comprising the freshening composition of claim 18, wherein the freshening product comprises the freshening composition in a spray dispenser.
